(19) United States
(12) Patent Application Publication
(10) Pub. No.: US 2021/0339033 A1
(43) Pub. Date: Nov. 4, 2021

(54) TREATMENT APPARATUS

(71) Applicant: Candela Corporation, Marlborough, MA (US)

(72) Inventors: Dany Berube, Milpitas, CA (US); Dmitry Gregoriev, Framingham, MA (US)

(73) Assignee: Candela Corporation

(21) Appl. No.: 17/240,356

(22) Filed: Apr. 26, 2021

Related U.S. Application Data
(60) Provisional application No. 63/017,112, filed on Apr. 29, 2020.

(51) Int. Cl. A61N 1/40

(52) U.S. Cl. CPC A61N 1/40

(57) ABSTRACT

A treatment apparatus system includes a plurality of needles configured to provide radio frequency energy, a source of radio frequency energy for supplying radio frequency energy to the plurality of needles, and a driving unit configured to insert the plurality of needles to different dermal depths. A user interface is configured to allow a user to select a plurality of depths the needles are to be inserted and, at each depth, an energy level to be applied. A controller subsystem is responsive to a user selected plurality of depths and a user selected energy level at each depth and is configured to control the driving unit to insert the plurality of needles to the user selected plurality of depths and, at each depth, control the source of radio frequency energy to apply the user selected energy level. A computer safety routine is configured to limit a user selected energy level when a user selected depth is less than a predetermined minimum depth from the skin surface.

### RELATED APPLICATIONS

This application claims benefit of and priority to U.S. Provisional Application Ser. No. 63/017,112 filed Apr. 29, 2020, under 35 U.S.C. §§ 119, 120, 363, 365, and 37 C.F.R. § 1.55 and § 1.78, which is incorporated herein by this reference.

### FIELD OF THE INVENTION

This subject invention relates to skin treatment devices and methods.

### BACKGROUND OF THE INVENTION

An array of radio frequency needles injected into the patient's dermis to treat wrinkles and to provide other treatments is shown in U.S. Pat. No. 9,364,392 incorporated herein by this reference.

### BRIEF SUMMARY OF THE INVENTION

It is often desirable for the user to be able to select the needle insertion depth(s) and also the energy level applied by the needles at each depth based on the purpose of the procedure, the skin characteristics of a given patient, and/or the user's experience and preferences.

Some users, however, may lack the requisite experience and/or select needle insertion depths and/or energy levels improperly.

We have discovered, for example, that if the needles are inserted to a depth of 1.5 mm or less from the skin surface and an energy level of over 2 Joules is applied, the result, in some conditions, can be the creation of an undesirable thermal injury to the skin surface. In other conditions, charring can result along the active portion of the needle and impede the delivery of RF energy.

Accordingly, in one embodiment herein, a safety routine limits the user's choice of energy levels when a user sets a needle depth less than a predetermined depth (e.g., 1.5 mm) from the skin surface.

Moreover, for some treatments, it is desirable to create thermal injuries in the subcutaneous or dermal area to partially denature collagen as the most optimal way to trigger a vigorous healing response and to maximize the clinical result. Too low of a thermal dose and the collagen is unaffected. Too high of a thermal dose and the collagen is fully denatured.

Accordingly, in one embodiment, partially denatured collagen is better assured via a default energy level setting corresponding to 2 Joules of applied energy to the subcutaneous or dermal area at each depth to partially denature the collagen therein thus triggering a vigorous healing response and maximizing the result. In this way, if the user is inexperienced and/or unsure of the optimal energy level to be applied at each needle insertion depth, the user does not need to change the default setting and yet the maximum effective energy levels are always applied by the system.

Featured is a treatment apparatus system including a plurality of needles configured to provide radio frequency energy, a source of radio frequency energy for supplying radio frequency energy to the plurality of needles, a driving unit may be configured to insert the plurality of needles to different dermal depths. A user interface is configured to allow a user to select a plurality of depths the needles are to be inserted and, at each depth, an energy level to be applied. A controller subsystem, is responsive to a user selected plurality of depths and a user selected energy level at each depth and is configured to control the driving unit to insert the plurality of needles to the user selected plurality of depths and, at each depth, control the source of radio frequency energy to apply the user selected energy level. A computer safety routine is configured to limit a user selected energy level when a user selected depth is less than a predetermined minimum depth from the skin surface. The safety routine may be configured to limit user selected energy per needle levels to no greater than 0.0408 Joules per needle when a user selected depth is 1.5 mm or less from the skin surface.

The user interface may further be configured to display and select a default energy per needle level corresponding to 0.0408 Joule per needle or approximately 0.0408 Joule per needle of applied energy per needle at each depth for optimally creating thermal injuries in the subcutaneous or dermal area partially denaturing collagen therein.

In one embodiment, the controller subsystem is configured to control the source of radio frequency energy to apply pulses of RF energy to the plurality of needles according to a duty cycle specific to each user selected energy level where the duty cycle is adjusted based on the real time integration of energy delivered to ensure precise RF energy delivery. The system may further include a needle depth measurement subsystem configured to measure the depth the needles are inserted and then controller subsystem is responsive to the needle depth measurement subsystem to control the driving unit to insert the plurality of needles to the user selected plurality of depths. The controller subsystem may be configured to control the driving unit to automatically insert the plurality of needles first to a user selected deepest depth and then to withdraw the needles to any user selected shallower depths in sequence. The needles may be 34 gauge needles. The controller subsystem, in one example, is configured to control the driving unit to insert the plurality of needles in response to a user selected first depth to a depth greater than the first user selected first depth and then to retract the needles to the user selected first depth. The controller subsystem may be further configured to limit the application of radio frequency energy to the needles so that any two needle insertions depths are no less than 0.8 mm apart when energy is applied to the needles.

Also featured is a treatment method including allowing a user to select, via a user interface, a plurality of depths the needles are to be inserted and, at each depth, an energy level to be applied. In response to a user selected plurality of depths and a user selected energy level at each depth, a driving unit is controlled to insert the plurality of needles to the user selected plurality of depths and, at each depth, the source of radio frequency energy is controlled to apply the user selected energy level. In the method a user selected energy level is automatically limited when a user selected depth is less than a predetermined minimum depth from the skin surface.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic view of an exemplary treatment apparatus;

FIG. 2 is a block diagram depicting the primary components associated with an exemplary treatment apparatus;

FIG. 3 is a flowchart showing the primary steps associated with an exemplary treatment method and also depicting the primary steps associated with the programming of the controller subsystem of FIG. 2;

FIG. 4 is an example of a user interface displaying default needle insertion depth levels and a default energy level at each depth;

FIG. 5 is another view of the user interface showing how the user is allowed to change one or more default needle insertion depths and/or one or more default energy levels;

FIG. 6 is another view of the user interface showing how, when a user selects a needle insertion depth less than a predetermined minimum, the safety routine of FIG. 3 limits the energy level at that depth to a preselected maximum;

FIG. 7 shows another example of the user interface where the user has selected a needle insertion depth less than the predetermined minimum but is allowed to select an energy level at that depth less than the predetermined maximum;

FIG. 8 shows an exemplary needle array;

FIG. 9 depicts an example of a needle for the array of FIG. 8; and

FIGS. 10A and 10B depict exemplary pulse cycles.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless them is clear and convincing evidence manifesting a certain exclusion, restriction, or disclaimer.

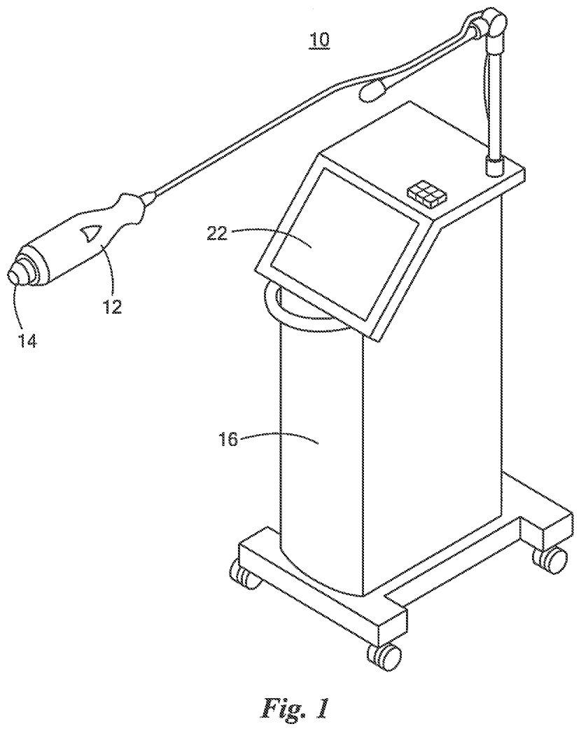

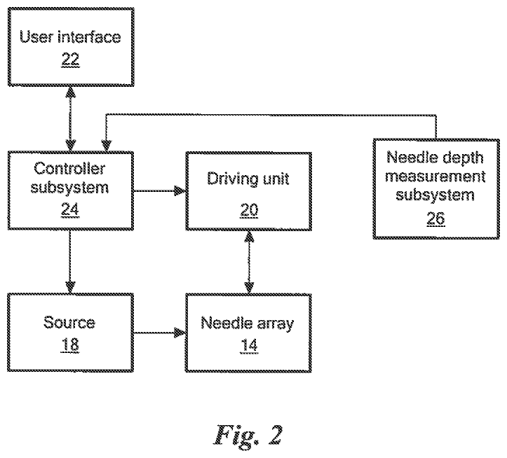

An exemplary treatment apparatus 10, FIG. 1 includes handpiece 12 with a plurality of needles 14, typically in an array, configured to provide radio frequency to energy to the patient's dermis. Console 16 preferably includes a source of radio frequency energy 18, FIG. 2 for supplying radio frequency energy to the plurality of needles. Handpiece 12, FIG. 1 includes a driving unit 20, FIG. 2, configured to insert the plurality of needles to different dermal depths. See U.S. Pat. No. 9,364,392 incorporated herein by this reference.

User interface 22, FIG. 1 (e.g., a touch screen) is configured to allow a user to select a plurality of depths the needles are to be inserted and at each depth an energy level to be applied. Console 16 may also house a controller subsystem 24, FIG. 2, responsive to user selected needle insertion depths and a user selected energy level at each depth. The controller subsystem may include one or more processors, one or more memories, instructions stored in the one or more memories and executed by the one or more processors, an application specific integrated circuit, a microcontroller, a field programmable gate array, or the like. The controller subsystem and its associated software may control the user interface and read values input into the user interface and/or may be distributed as between the user interface and a computer unit. Typically, computer instructions are stored in memory and are executed by one or more processors to control the driving unit to insert the plurality of needles to the user's selected plurality of depths. At each depth, the computer instructions control the source of radio frequency energy to apply the user selected energy level.

The computer instructions also include a computer safety routine configured to limit user selected energy levels when a user selected depth is less than a predetermined minimum depth from the skin surface. In one embodiment where the electrode array, for example, consists of 7×7 needle assembly (for a total of 49 needles), the safety routine is configured to limit the user selected energy levels to no greater than 2 Joules or a value corresponding to 2 Joules applied to the patient's dermis when a user selected depth is 1.5 mm or less from the skin surface. The computer instructions are also preferably configured so that the user interface displays and selects a default energy level preferably corresponding to 2 Joules, and more generally between 1.5 and 2.5 J, or approximately 2 Joules applied energy at each depth for optimally creating thermal injuries in subcutaneous or dermal area partially denaturing collagen therein. The user interface may be controlled by controller subsystem 24, FIG. 2 to display and select these default energy levels. To generalize the concept, it is useful to normalize the total energy by the energy per needle, so the principle can be expanded to other array sizes. Indeed, one normally skilled in the art would appreciate that the energy per needle is important to generalize the concept. In the example above, the energy per needle would be calculated by dividing the total energy by the total of needles as follow: 2 J/49 needles=0.0408 J/needle. Therefore, using the same energy per needle value, the same results and characteristics described above using 2 J for a 49-needle array would be the same as using 0.0408 J/needle*25 needles=1.02 J for a 5×5 needle array. In fact, all energy levels presented in this document have been obtained with the preferred embodiment using a 49 needles array (7×7). Therefore, all energy values in this document could be divided by 49 to calculate the corresponding energy per needle (as illustrated above), in order to generalize the concept.

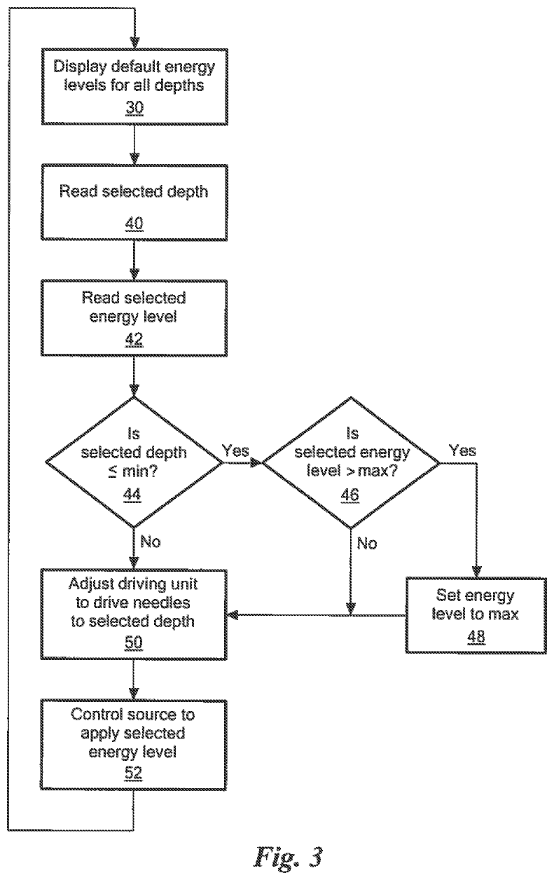

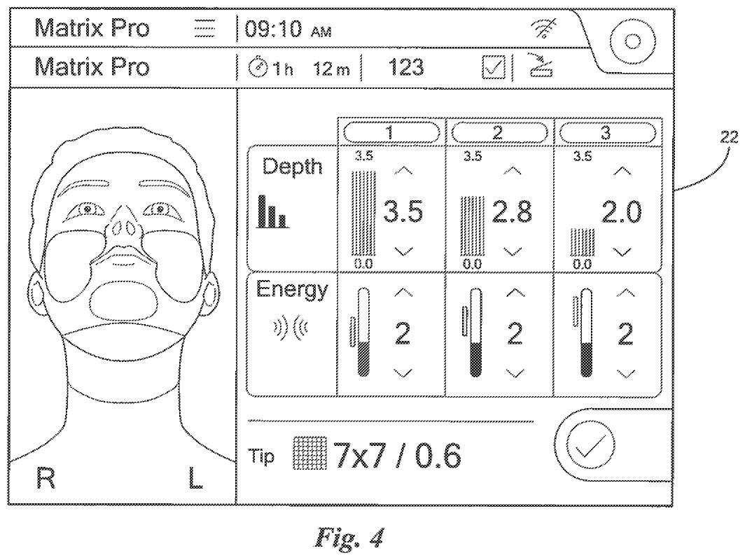

As shown in FIG. 3, the controller subsystem 24, FIG. 2 is configured to display a default energy level for all depths, step 30, FIG. 3. Thus, as shown in FIG. 4, in one example, the user interface, upon startup of the system, displays a default energy level of “2” corresponding to 2 Joules of energy applied to the patient's dermis at each depth and, optionally, needle insertion default depth levels of 3.5 mm, 2.8 mm, and 2.0 mm from the skin surface. In another embodiment, the selectable energy levels may be, for example, “high” (corresponding, for example, to an energy level of 3-4 Joules of energy applied to the patient's dermis), “medium” (corresponding, for example, to 2 Joules of energy applied to the patient's dermis), and “low” (corresponding, for example, to 1 Joule of energy applied to the patient's dermis). In this example, the displayed default energy level at each depth would be “medium”. As described above, these values were obtained with a preferred needle array embodiment of 49 needles (7×7) and can be generalized by calculating the energy per needle, as described earlier. The “high”, “medium”, and “low” levels could also be defined as range of values. For example, “high” would correspond to a range between 3 and 4 J, “medium” would correspond to a range between 2 and 3 J, and “low” would correspond to a range between 1 and 2 J.

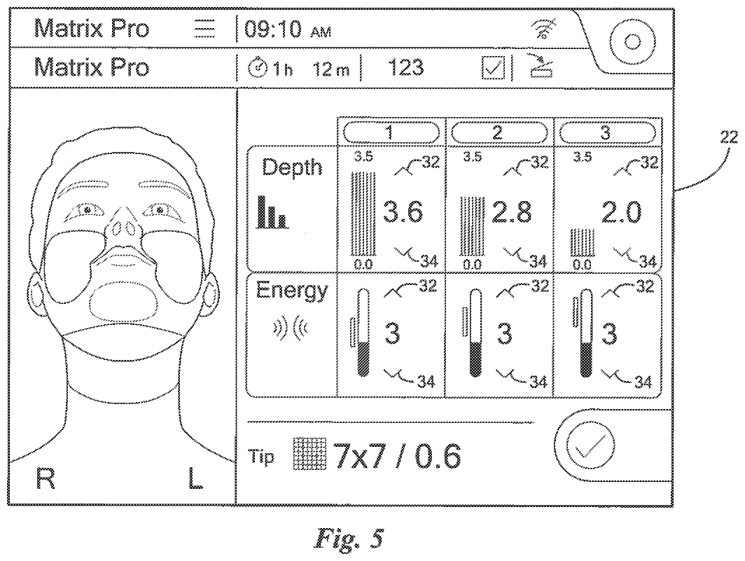

As shown in FIG. 5, by pressing up 32 and down 34 arrows, the user can conveniently change the default depth selections and default energy levels. Thus, in FIG. 5, the user has selected a first depth of 3.6 mm and an energy level of 3 (corresponding, for example, to 3 Joules of energy applied to the patient's dermis) for all three depths. The default depths for the intermediate depth and the shallow depth have not changed by the user.

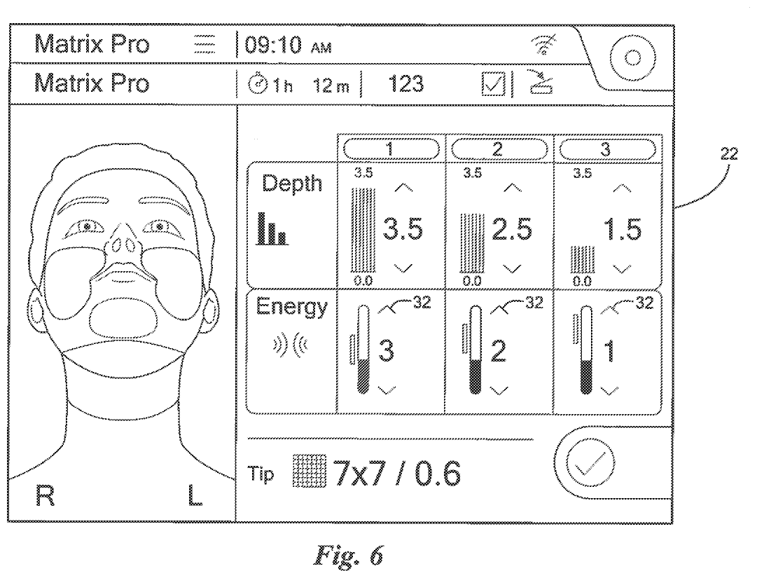

But, as shown in FIG. 6, when the user selects a depth of 1.5 mm (or less) (or some other predetermined minimum depth from the skin surface) for the third depth and then attempts to change the default energy level of “2” to a higher level, the safety routine will not allow the user to use up arrow 32 to increase the energy level above 2. The safety routine may also cause the display to flash, to display a message that a higher energy level is not available for needle insertion depths of less than or equal to 1.5 mm or sound an audible alarm.

FIG. 6 also shows that for deeper needle insertion depths (e.g., 3.5 mm and 2.5 mm), the user is allowed to use up arrows 32 to increase the energy level applied above the default level of 2 to 3 Joules and 4 Joules, respectively.

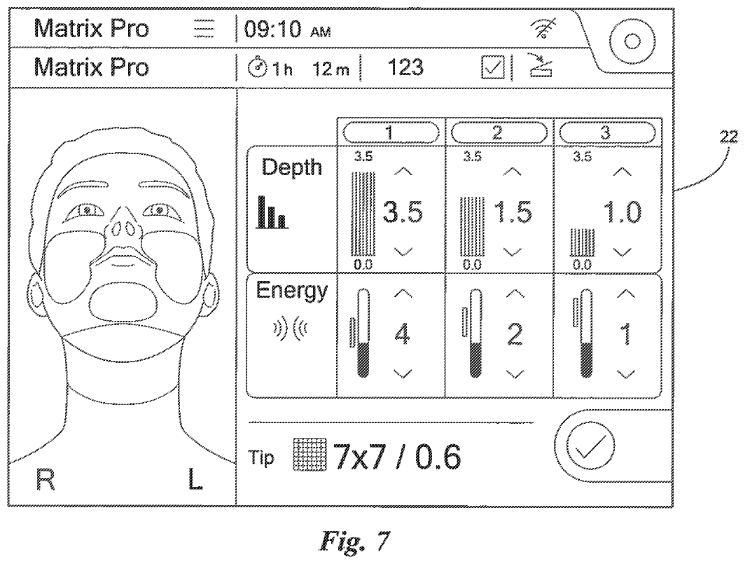

As shown in FIG. 7, the user has selected, for the third needle insertion depth, a value of 1.0 mm and an energy level of 1 Joule which is allowed at this depth by the computerized safety routine. The same routine, however, would not allow the user to select an energy level higher than 2 Joules for a needle insertion depth of 1.0 mm. Thus, the computerized safety routine insure no undesirable thermal injuries are created on the skin surface for shallow needle insertion depths, or in case of isolation layer loss along the needles.

Thus, controller subsystem 24, FIG. 2 reads each selected needle insertion depth, step 40, FIG. 3 and reads the selected energy level at each depth, step 42. If any selected depth is less than or equal to a predetermined minimum (e.g., 1.5 mm), step 44, and the selected energy level is greater than a preselected maximum energy level (e.g., 2 Joules), step 46, the safety routine limits the energy level, step 48 to a predetermined energy level (e.g., 2 Joules). The controller subsystem then adjusts the driving unit 20, FIG. 2 to drive the needles 14 to the selected insertion depths, step 50, FIG. 3 and, at each depth, to control the source 18, FIG. 2 to apply the selected energy level at each depth, step 52.

The controller subsystem is preferably configured to control the source of radio frequency energy by using a duty cycle technique in order to deliver the amount of energy selected by the user. The system may further include a needle depth measurement subsystem 26, FIG. 2 configured to measure the depth the needles are inserted and controller subsystem 24 is responsive to the needle depth measurement subsystem to control the driving unit to insert the plurality of needles to the user selected plurality of depths. See U.S. Pat. No. 9,364,392 incorporated herein by this reference. Preferably, the controller subsystem is configured to control the driving unit to automatically insert the plurality of needles first to a user selected deepest depth and then to withdraw the needles to any user selected shallower depths in sequence.

Preferably, the RF-needles apply short pulses of RF energy to create small zones of fractional thermal injures at the needle tips. One of the advantages of using short pulses is pain management since topical anesthesia is sometimes sufficient to properly manage patient's discomfort. Compared to other RF-needle based platform designed to create much larger zones of fractional injuries in dermis (such as the system described in U.S. Pat. No. 8,540,705, for example), the anesthetic procedure is much easier to perform, and can easily be delegated to nurses, estheticians, or other non-physician staff. Another advantage is the procedural easiness since the array of needles goes straight down in skin without much need to manipulate the skin in order to insure proper needle penetrations.

Some commercially available platforms, for example the Infini system commercialized by Lutronic), are also capable of adjusting the needle penetration depth to target different dermal layers. A typical procedure consists of performing multiple passes (usually three) covering the entire treatment area, at different dermal depths, but one depth at the time. To produce thermal injuries on more than one depth, the user generally has to go back to the treated area, set another depth, and retreat the same area which increases the overall procedural duration. The system described herein is capable of creating multiple zones of fractional thermal injuries, at different depths, for each needle insertion to decrease the overall procedural duration and to avoid retreating the same treatment area multiple times. Ideally, all the desired thermal injury depths would be performed in one needle insertion, so the entire procedure would be completed in a single pass covering the entire treatment area.

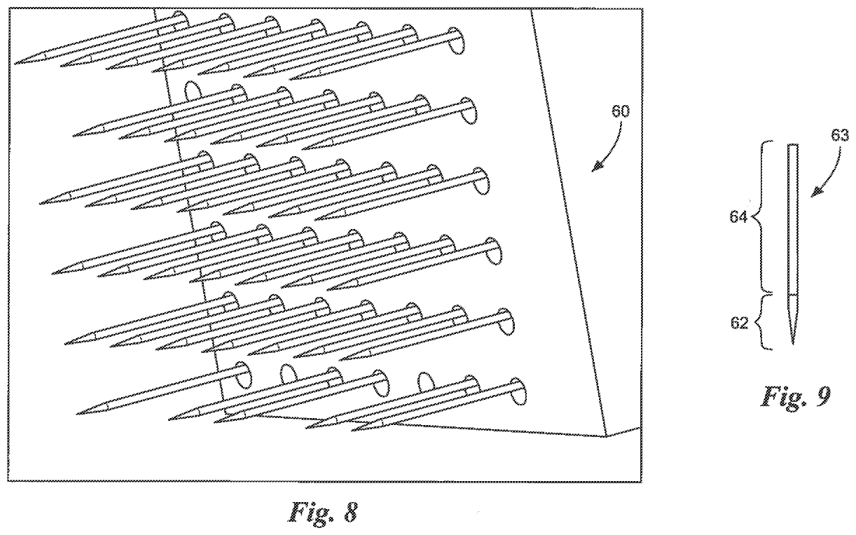

In one embodiment, the needle array 60, FIG. 8 includes 49 needles with a diameter of 34 Ga (0.16 mm), arranged in a 7×7 configuration covering an area of 1 cm². The electrically active part 62, FIG. 9 of the needle 63 is preferably located at the distal end, where 0.6 mm of metal is exposed to allow the deposition of RF energy (the metallic part at the distal end of the needles in. The most proximal part 64 of the needles are covered with a thin layer of Teflon. In one embodiment, the user will be capable of using the system to create zones of thermal injuries in up to 3 planes, with the deepest plane located at a maximal depth of up to 3.5 mm underneath the skin surface.

After the user activates a trigger allowing the needle insertion in skin, the control software and handpiece will activate the needle array to insert the needle tips slightly beyond the deepest point first, and then back to the desired location. This is usually done to compensate for the tenting effect of the skin when an array of needles is inserted and to increase the needle tip placement accuracy. After the deepest point is reached, the system will allow the RF source to deliver the amount of energy selected by the user for that specific depth to create a series of thermal injuries at the tip of the needles. Then, the needle array will be pulled back to the adjacent shallower depth (if necessary) and the process will be repeated.

It is generally desirable to keep the army of thermal injuries fractional in order to optimize the healing process and minimize healing time. To do so, it is preferable to configure the system in such a way to keep a zone of healthy tissue surrounding each thermal injury. Therefore, it is not desirable to create a long column of thermal injury along the penetration line of a needle. Consequently, a minimum distance between the closest points of two collinear adjacent thermal injuries should be kept to insure fractionality. In one system, the minimum distance is set to one third (⅓) of the active length of the needle. If, for example, the active length is 0.6 mm, the minimum distance is 0.2 mm. The minimum distance between two adjacent thermal injuries is therefore 0.8 mm, center to center. A minimum distance of one third of the active needle length is also kept between the skin surface and the proximal part of the most superficial thermal injury to minimize the risks of a thermal injury on the skin surface.

Finite Element Analysis (FEA) was used to select the energy levels which will be allowed by the system. For a device using a 7×7 array of 0.6 mm long active needles, the RF energy absorbed in tissue will be 1, 2, 3, or 4 Joules. To maximize safety and insure no undesirable thermal injuries are created on the skin surface in case of incomplete needle insertion at shallow depths, or in case of isolation layer loss along the needles, the maximal allowable energy is limited to 2 J when the needle depths are within 1.5 mm underneath the skin surface.

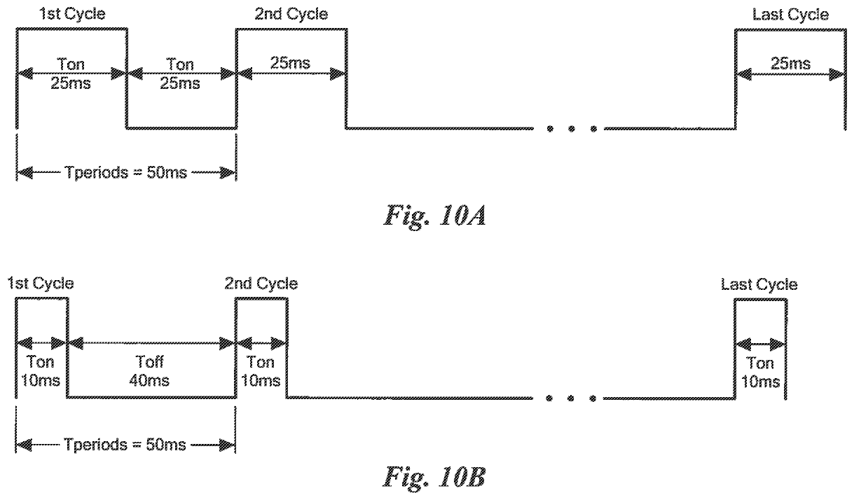

In an exemplary algorithm to deliver the precise amount of RF energy in tissue, the first step is to use a low energy RF pre-pulse to measure the tissue impedance measured by the array of needles. Then, a duty cycle value of the RF pulse is adjusted to obtain an average power of 10 W to deliver exactly 0.5 J every 50 ms. A duty cycle is necessary if the RF source used to supply the RF power to the electrode array has a minimum power of more than 10 W. A duty cycle may be used to decrease the average power to the desired 10 W. Since a specific amount of energy is delivered in a specific amount of time, the total RF pulse length varies with the user-selected energy to be delivered. The possible RF pulse durations are 100, 200, 300, or 400 ms for 1, 2, 3, or 4 J, respectively. In another example, the maximum output energy was limited to 4 J by 1 J step increases. The maximum output peak power is 50 W±20%, the minimum output peak power is 20 W±20%. The duty cycle is 50% or less. The minimum pulse on time is 10 ms and the maximum pulse on time is 25 ms. The minimum number of pulses is 2 and the maximum number of pulses is 8. The average power may always be 10 W, regardless of the impedance conditions. The period of the pulse cycle is preferably 50 ms. Then, the shortest pulse (when a pulse is stopped) is <3 ms. 1, 2, 3, or 4 J is delivered with ±20% accuracy. Exemplary pulse cycles are shown in FIGS. 10A and 10B.

A measured impedance for the first measured point of the first cycle can be used to recognize any poor needle coupling. Impedance is measured approximately every 2 ms during a pulse for energy accumulation:

$${\sum }_{N = 1}^{N} E = {P}_{1} * 2 ms + {P}_{2} * 2 ms + ……… + {P}_{n} * 2 ms \qquad ( 1 )$$

The pulse is stopped if a bad coupling is recognized.

In general, two situations are available: 1) the accumulated energy reaches 90% and more energy is required in which case the pulse is stopped and pulsing continues to the next cycle and/or 2) the accumulated energy reaches 90% and more of the required energy at the last pulse and then the pulse is stopped as is any further treatment for this step. All of these steps may be repeated and N pulse times.

Preferably a single pulse is broken into subpulses, depending on the energy selected on the GUI. Each subpulse deliver 0.5 J, so 4 J would require 8 subpulses. In effect the RF pulse is stretched so that the average RF power is 10 W to avoid too rapid heating that causes more uneven heating and smaller damage zones.

Creating thermal injuries in dermis to partially denature collagen is usually the most optimal way to trigger a vigorous healing response and maximize the clinical results. If the total RF pulse is within 0.4 sec, the desired dermal temperature levels necessary to partially denature collagen would be between about 66° C. and about 73° C., as described in FIG. 5 of the paper by Alexiades et al., entitled: “Randomized, bylined, 3-arm clinical trial assessing optimal temperature and duration for treatment with minimally invasive fractional radiofrequency” (Dermatol Surg 2015; 41:623-632.).

FEA analysis was used to determine the energy levels required to produce such conditions in dermis. It was shown that a delivered energy of 2 J would be defined as the preferred default setting to maximize the volume of simulated dermis where the temperature levels are between 66 and 73° C.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A treatment apparatus system comprising:
a plurality of needles configured to provide radio frequency energy;
a source of radio frequency energy for supplying radio frequency energy to the plurality of needles;
a driving unit configured to insert the plurality of needles to different dermal depths;
a user interface configured to allow a user to select a plurality of depths the needles are to be inserted and, at each depth, an energy level to be applied;
a controller subsystem, responsive to a user selected plurality of depths and a user selected energy level at each depth and configured to:
control the driving unit to insert the plurality of needles to the user selected plurality of depths and, at each depth, controlling the source of radio frequency energy to apply the user selected energy level; and
a computer safety routine configured to limit a user selected energy level when a user selected depth is less than a predetermined minimum depth from the skin surface.

2. The system of claim 1 in which the safety routine is configured to limit user selected energy per needle levels to no greater than 0.0408 Joules per needle when a user selected depth is 1.5 mm or less from the skin surface.

3. The system of claim 1 in which the user interface is configured to display and select a default energy per needle level corresponding to 0.0408 Joule per needle or approximately 0.0408 Joule per needle of applied energy per needle at each depth for optimally creating thermal injuries in the subcutaneous or dermal area partially denaturing collagen therein.

4. The system of claim 1 in which the controller subsystem is configured to control the source of radio frequency energy to apply pulses of RF energy to the plurality of needles according to a duty cycle specific to each user selected energy level where the duty cycle is adjusted based on the real time integration of energy delivered to ensure precise RF energy delivery.

5. The system of claim 1 further including a needle depth measurement subsystem configured to measure the depth the needles are inserted and the controller subsystem is responsive to the needle depth measurement subsystem to control the driving unit to insert the plurality of needles to the user selected plurality of depths.

6. The system of claim 1 in which the controller subsystem is configured to control the driving unit to automatically insert the plurality of needles first to a user selected deepest depth and then to withdraw the needles to any user selected shallower depths in sequence.

7. The system of claim 1 in which the needles are 34 gauge needles.

8. The system of claim 1 in which the controller subsystem is configured to control the driving unit to insert the plurality of needles in response to a user selected first depth to a depth greater than the first user selected first depth and then to retract the needles to the user selected first depth.

9. The apparatus of claim 1 in which the controller subsystem is further configured to limit the application of radio frequency energy to the needles so that any two needle insertions depths are no less than 0.8 mm apart when energy is applied to the needles.

10. A treatment method comprising:
allowing a user to select, via a user interface, a plurality of depths the needles are to be inserted and, at each depth, an energy level to be applied;
in response to a user selected plurality of depths and a user selected energy level at each depth;
controlling a driving unit to insert the plurality of needles to the user selected plurality of depths and, at each depth, controlling a source of radio frequency energy to apply the user selected energy level; and
automatically limiting a user selected energy level when a user selected depth is less than a predetermined minimum depth from the skin surface.

11. The method of claim 10 in which the user selected energy per needle levels are limited to no greater than 0.0408 Joules per needle when a user selected depth is 1.5 mm or less from the skin surface.

12. The method of claim 10 including displaying and selecting on the user interface a default energy per needle level corresponding to 0.0408 Joule per needle or approximately 0.0408 Joule per needle of applied energy per needle at each depth for optimally creating thermal injuries in the subcutaneous or dermal area partially denaturing collagen therein.

13. The method of claim 10 further including controlling a source of radio frequency energy to apply pulses of RF energy to the plurality of needles according to a duty cycle specific to each user selected energy level.

14. The method of claim 10 further including measuring the depth the needles are inserted and inserting the plurality of needles to the user selected plurality of depths based on the measured depths where the duty cycle is adjusted based on the real time integration of energy delivered to ensure precise RF energy delivery.

15. The method of claim 10 including automatically inserting the plurality of needles first to a user selected deepest depth and then withdrawing the needles to any user selected shallower depths in sequence.

16. The method of claim 10 in which the needles are 34 gauge needles.

17. The method of claim 10 further including controlling the driving unit to insert the plurality of needles in response to a user selected first depth to a depth greater than the first user selected first depth and then to retract the needles to the user selected first depth.

18. The method of claim 10 further including limiting the application of radio frequency energy to the needles such that at any two insertions depths are no less than 0.8 mm apart when energy is applied to the needles.
